(19) United States
(12) Patent Application Publication
(10) Pub. No.: US 2021/0389235 A1
(43) Pub. Date: Dec. 16, 2021

(54) GAS MEASUREMENT DEVICE AND GAS MEASUREMENT METHOD

(71) Applicant: Shimadzu Corporation, Kyoto (JP)

(72) Inventor: Kazune MANO, Kyoto (JP)

(21) Appl. No.: 17/285,410

(22) PCT Filed: Nov. 20, 2018
(86) PCT No.: PCT/JP2018/042825

§ 371 Date: Apr. 14, 2021

(51) Int. Cl. G01N 21/17; G01N 21/31; G01N 33/00

(52) U.S. Cl. CPC G01N 21/1717; G01N 21/31; G01N 2201/023; G01N 2201/06113; G01N 33/004

(57) ABSTRACT

After a measurement-target gas has been introduced into a measurement cell (40) to a predetermined pressure, a measurement by CRDS at a predetermined wavenumber is performed using a laser source unit (1), optical switch (3), optical resonator (4) and photodetector (5). A portion of the measurement-target gas is subsequently discharged from the measurement cell (40) to lower the pressure, and a measurement at the wavenumber of an absorption peak of the target component ¹⁴CO₂ is performed. Since the influence of the absorption by ¹⁴CO₂ in the measurement at high pressure is negligible, the concentration of the background, including ¹²CO₂, can be determined from a ring-down time determined in this measurement. An absorption coefficient calculated from a ring-down time determined from measurement data acquired at low pressure contains an influence of the background, while the absorption coefficient of the background at low pressure can be determined from the concentration of the background determined at high pressure. Using this absorption coefficient, a concentration-computing operator (73) determines the absorption coefficient of only ¹⁴CO₂ which is free from the influence of the background, and calculates the concentration of only ¹⁴CO₂. Thus, based on the results of the two measurements performed at different pressures, an accurate absolute concentration of a target component, such as ¹⁴CO₂, can be obtained. The measurement time can be shortened as compared to a conventional case.

### TECHNICAL FIELD

The present invention relates to a gas measurement device and gas measurement method for measuring the concentration of a specific component in a gas to be subjected to a measurement (this gas is hereinafter called the “measurement-target gas”) by using the absorption of laser light by the gas.

### BACKGROUND ART

Laser absorption spectroscopy has been widely used as a technique for measuring the concentration of a specific component in a measurement-target gas. There are several known types of laser absorption spectroscopy, one of which is cavity ring-down absorption spectroscopy (which is hereinafter abbreviated as “CRDS” according to a common practice). CRDS is a technique in which the effective optical path length for the absorption of light is increased by using an optical resonator, whereby the degree of detectable absorbance, or detection sensitivity, can be dramatically improved (see Non Patent Literature 1 or other related documents).

### CITATION LIST

### Patent Literature

Patent Literature 1: JP 2011-119541 A
Patent Literature 2: JP 2018-4656 A

### Non Patent Literature

Non Patent Literature 1: Koji Hashiguchi, “A survey on high-efficiency measurement techniques of trace moisture in gases”, National Institute of Advanced Industrial Science and Technology (AIST), AIST Bulletin of Metrology, October 2015, Vol. 9, No. 2, pp. 185-205
Non Patent Literature 2: Yaeko Suzuki, “Hydrogen and Oxygen stable isotope analysis of water in fruits and vegetables by using cavity ring-down spectrometry”, Journal of Japanese Association of Hydrological Sciences, 2016, Vol. 46, No. 2, pp. 157-166
Non Patent Literature 3: Pan Du and two other authors, “Improved peak detection in mass spectrum by incorporating continuous wavelet transform-based pattern matching”, Oxford University Press, Bioimformatics, 2006, Vol. 22, No. 17, pp. 2059-2065

### SUMMARY OF INVENTION

### Technical Problem

A problem to be solved by the present invention is hereinafter described with reference to the drawings.

FIG. 8 is a schematic configuration diagram of a commonly used CRDS device. In FIG. 8, a predetermined wavelength of laser light emitted from a laser source unit 1 is introduced through an optical switch 3 into a measurement cell 40 which contains a measurement-target gas. The tubular measurement cell 40 has a pair of high-reflection mirrors 47 and 48 (which allow a minute amount of light to pass through) arranged at both ends of the cell and facing each other. The measurement cell 40 and those mirrors 47 and 48 constitute an optical resonator 4. This optical resonator 4 is, for example, a Fabry-Perot resonator similar to those commonly used in laser devices or the like. The wavelength (frequency) of light which can resonate is determined by resonance conditions. A ring-type resonator formed by three or more mirrors may also be used as the optical resonator 4 in place of the resonator having two mirrors facing each other.

Frequencies at which resonation can occur in the optical resonator 4 are generally called “mode frequencies”. As shown in FIG. 9, the mode frequencies exist at predetermined intervals of frequency. No optical power will be accumulated in the optical resonator 4 when the frequency of the laser light introduced into the optical resonator 4 is not equal to any of the mode frequencies. When the laser oscillation frequency in the laser source unit 1 is regulated so that it becomes equal to one of the mode frequencies, optical power will be accumulated in the optical resonator 4.

In the CRDS device, after a sufficient amount of optical power has been accumulated in the optical resonator 4, the laser light entering the optical resonator 4 is instantaneously blocked by the optical switch 3. The light which has been accumulated in the optical resonator 4 until immediately before the blocking travels back and forth between the pair of mirrors 47 and 48 a considerable number of times (actually, thousands to tens of thousands of times). During this travel, the light is gradually attenuated due to the absorption by the components in the measurement-target gas contained in the measurement cell 40. Meanwhile, a portion of the light leaks through one mirror 48 of the optical resonator 4 to the outside. This light is repeatedly detected with a photodetector 5 to determine the state of attenuation of the light. Based on the data acquired with the photodetector 5, the time constant of the optical attenuation (ring-down time) is determined, from which the absorption coefficient of the target component in the measurement-target gas at the frequency of the laser light at that moment can be calculated. From this absorption coefficient, the absolute concentration of the target component can be determined. Furthermore, an absorption spectrum of the target component in the measurement-target gas can also be acquired by repeating similar measurements while continuously varying the laser oscillation frequency in the laser source unit 1.

In order to determine absorption coefficient α of a target component in a measurement-target gas, the following equation (1) is normally used (see Patent Literature 1 or other related documents):

α=1/c{(1/τ)−(1/τ₀)} (1)

where c is the speed of light, τ is the ring-down time under the condition that the measurement-target gas is contained in the measurement cell 40, and τ₀ is the ring-down time under the condition that the measurement-target gas is not contained in the measurement cell 40 (e.g., the cell is in the state of vacuum), or under the condition that the absorption by the components in the measurement-target gas is entirely negligible. The absorption coefficient α, number density n and absorption cross section a of a target component (absorbing substance) satisfies a relationship expressed by the following equation (2):

α=nσ (2)

Accordingly, by using equations (1) and (2), the absolute concentration for a component whose absorption cross section is previously known can be calculated from the ring-down times τ and τ₀. In the CRDS device, since the effective distance which the light travels through the measurement-target gas is increased by the optical resonator 4, a substantial difference occurs between the ring-down times τ and τ₀. Therefore, even an infinitesimal amount of optical absorption by a trace amount of target component can be detected, so that a high level of detection sensitivity can be achieved as compared to other types of laser absorption spectroscopy.

As just noted, a CRDS device can measure the concentration of a component in a measurement-target gas with an extremely high level of sensitivity. Accordingly, CRDS devices are often used for a high-accuracy measurement of an isotopic ratio of CO₂ or H₂O in a measurement-target gas (see Patent Literature 2 or other related documents). Measurements of isotopic ratios using CRDS devices have been increasingly applied in various areas, such as the identification of the place of origin of agricultural products (see Non Patent Literature 2).

However, as is the case with ¹⁴CO₂ containing a radioactive isotope of carbon, ¹⁴C (natural isotope abundance ratio: 1×10⁻¹²), when the concentration of a component whose content in the measurement-target gas is extremely low needs to be measured, it is impossible to ignore the background, i.e., the influence of the absorption by other components in the measurement-target gas. FIG. 10 schematically shows an absorption spectrum of a measurement-target gas around a peak wavelength of an absorption line of ¹⁴CO₂. Actually, the baseline of this absorption peak is mostly composed of the background originating from the absorption by ¹²CO₂ and ¹³CO₂ which are contained at high concentrations. Disregarding this background means that the concentration of the target component cannot be accurately determined.

Accordingly, when the concentration of an isotope gas whose content is comparatively low needs to be measured with a high level of accuracy, the task of removing the background has conventionally been performed as follows:

While the laser oscillation wavelength is continuously varied over a predetermined range, a CRDS measurement is performed at each of a plurality of wavelengths around an absorption peak of an isotope gas which is the measurement target, and an absorption coefficient is calculated from each of the obtained measurement results. Based on the absorption coefficients respectively obtained for the different wavelengths, a fitting operation using a Voigt function or Lorentz function is performed to estimate the spectral waveform of the background (in the case of FIG. 10, the absorption by gas species other than ¹⁴CO₂). Specifically, in FIG. 10, the spectral waveform in section C, which corresponds to the baseline of the peak, is estimated from the spectral waveforms in sections A and B. Using the estimated spectral waveform, the background removal is performed to determine the absorption coefficient of only the isotope gas which is the measurement target, and the concentration is calculated from the absorption coefficient.

In order to accurately perform the fitting for the spectral waveform of the background, it is necessary to determine absorption coefficients at a considerable number of wavelengths by measurements. Therefore, it is necessary to perform measurements a large number of times for one measurement-target gas, so that the measurement time will be considerably protracted. Needless to say, the estimation of the spectral waveform of the background must be performed every time the measurement-target gas is changed.

During the measurement, the effective reflectance of the mirrors may decrease due to the adsorption of some components in the measurement gas. A change in the effective reflectance or resonator length may also occur due to a slight change in the position of the mirror or an infinitesimal shift of the incident position of the incoming light. Furthermore, a change in the resonator length or other related dimensions may occur due to a thermal expansion of the optical resonator caused by a slight change in temperature. Therefore, as the measurement time becomes longer, the measurement conditions are more likely to change in the middle of the measurement, and the amount of that change may be even greater. This may consequently prevent accurate determination of the concentration.

As will be hereinafter described, if the spectral waveform corresponding to the baseline estimated in the previously described manner is inaccurate, the concentration cannot be accurately determined even after the fitting has been performed. FIG. 11A shows computed absorption spectra showing the relationship between the wavenumber and absorption coefficient for ¹⁴CO₂ (mentioned earlier) as well as ¹²CO₂ and ¹³CO₂ which respectively contain stable isotopes of carbon, ¹²C and ¹³C, within a predetermined wavenumber range around the position (wavenumber) of the absorption peak of ¹⁴Co₂. FIG. 11B shows the result of a calculation of the degrees of contribution of the absorption by ¹²CO₂, ¹³CO₂ and ¹⁴CO₂ at the position of the absorption peak of ¹⁴CO₂.

As can be seen in FIG. 11A, in the present example, the base portion of the absorption peak of ¹³CO₂ overlaps the position of the absorption peak of ¹⁴CO₂. This problem is normally solved as follows: The wavenumber range for the measurement is widened to cover a spectral range where the absorption by ¹³CO₂ is singly present. Spectrum fitting is performed on each of the ¹⁴CO₂ and ¹³CO₂ spectra. The absorption peak of ¹³CO₂ overlapping the position of the absorption peak of ¹⁴CO₂ is separated and removed to isolate the absorption peak of ¹⁴CO₂. This peak is ultimately evaluated. However, if the measurement is exclusively performed on the absorption peak of ¹⁴CO₂ to improve the measurement throughput in the previously described manner, it is impossible to locate the absorption peak of ¹³CO₂ alone. Therefore, the spectrum fitting cannot be correctly performed on the absorption by ¹³CO₂, so that the spectral waveform corresponding to the estimated baseline will be inaccurate.

A spectrum determined by repeating a measurement by CRDS while continuously varying the oscillation wavelength will actually be a waveform as shown by the solid line in FIG. 10. A baseline estimated from this waveform cannot be a baseline that reflects the overlapping peaks as shown in FIG. 11A. Therefore, it is impossible to accurately remove the background, and the absorption coefficient of the target component cannot be accurately determined.

Thus, in the case where a plurality of components whose absorption peaks are located close to each other are present in the measurement-target gas, the signals corresponding to those components may possibly overlap each other in an absorption spectrum of the measurement-target gas and prevent the accurate determination of the absorption coefficient of the target component. A technique for separating overlapping signals from each other by image processing (the so-called “peak picking”; for example, see Non Patent Literature 3) can be used to deal with this type of problem. However, the accuracy of the signal separation by commonly used peak-picking techniques often depends on the skill and experience of the operator. Therefore, it is not always the case that the signal separation is accurately performed, and it is difficult to ensure the reliability and reproducibility of the measurement result.

The present invention has been developed in view of the previously described problem. Its objective is to provide a gas measurement device and gas measurement method which can determine an accurate absorption coefficient and concentration of a target component.

### Solution to Problem

As described in Patent Literature 2 and other related documents, the ring-down characteristics in CRDS (the extent of the exponential attenuation of the intensity of light) depend on temperature and pressure. Accordingly, the present inventor has paid attention to the fact that the ring-down characteristics, i.e., the absorption coefficient, changes depending on the pressure. Changing the temperature of the measurement-target gas would cause an unfavorable change in the optical path length of the optical resonator or in the reflectance of the mirrors due to the effect of thermal expansion, which would change the mode frequencies or mode linewidths of the optical resonator, making it difficult to perform the measurement in a stable manner. On the other hand, the pressure of the measurement-target gas can be varied comparatively easily and accurately. The present inventor repeated simulation calculations and the like, and ultimately discovered that the degree of absorption by the same component can be dramatically changed by varying the pressure within a practically feasible range. Thus, the present invention has been completed.

That is to say, in a gas measurement method for determining the concentration of a target component contained in a measurement-target gas by cavity ring-down absorption spectroscopy (CRDS), the gas measurement method according to the present invention developed for solving the previously described problem includes:

a first measurement step for performing a measurement by cavity ring-down absorption spectroscopy for the wavelength of an absorption peak of the target component under a first pressure by irradiating the measurement-target gas with laser light;

a second measurement step for performing a measurement by cavity ring-down absorption spectroscopy by irradiating the measurement-target gas under a second pressure different from the first pressure with laser light; and

a calculation step for calculating the concentration of the target component by performing a calculation on a measurement result of the first measurement step and a measurement result of the second measurement step.

The gas measurement device according to the present invention developed for the previously described problem is a device for carrying out the gas measurement method according to the present invention. In a gas measurement device configured to determine the concentration of a target component contained in a measurement-target gas by cavity ring-down absorption spectroscopy, the gas measurement device according to the present invention includes:

a laser light emitter;

an optical resonator including a measurement cell configured to contain a measurement-target gas, the optical resonator configured to produce oscillations of laser light emitted from the laser light emitter and introduced into the measurement cell;

a photodetector configured to detect laser light extracted from the optical resonator;

a pressure regulator configured to regulate the pressure of the measurement-target gas in the measurement cell;

a controller configured to control the pressure regulator when performing a measurement for the measurement-target gas in the measurement cell by cavity ring-down absorption spectroscopy; and

a calculation processor configured to calculate the concentration of the target component by performing a calculation on a plurality of measurement results respectively obtained at different pressures under the control of the controller.

In the gas measurement device according to the present invention, in order to carry out the gas measurement method according to the present invention, the controller may be configured to control the laser emitter and the photodetector in addition to the pressure regulator so as to perform:

a first measurement step for performing a measurement by cavity ring-down absorption spectroscopy for the wavelength of an absorption peak of the target component under a first pressure by irradiating the measurement-target gas with laser light; and

a second measurement step for performing a measurement by cavity ring-down absorption spectroscopy by irradiating the measurement-target gas under a second pressure different from the first pressure with laser light.

As noted earlier, CRDS is a suitable technique for detecting a low-concentration component in a measurement-target gas with a high level of sensitivity. Accordingly, in most cases, the “target component” in the present invention is a component contained in a measurement-target gas at a comparatively low concentration. A typical example is an isotope having a low content ratio among isotopes expressed by the same chemical formula, such as ¹⁴CO₂, DHO (heavy water) or ¹⁵HN₃.

For example, in a CRDS measurement for ¹⁴CO₂ which is one of the carbon isotopes of CO₂, i.e., when the target component is ¹⁴CO₂, the pressure condition of the measurement-target gas is normally determined so that the ratio of the absorption by ¹⁴CO₂ to that of the absorption by other isotopes, i.e., ¹²CO₂ and ¹³CO₂, will be maximized. In that case, the first pressure in the present invention is a pressure which satisfies the optimum (or nearly optimum) condition for the measurement of ¹⁴CO₂, and the CRDS measurement for the wavelength of the absorption peak of the target component is performed at this pressure. However, the wavelength of the absorption peak due to ¹³CO₂ is considerably close to that of the absorption peak due to ¹⁴CO₂. Therefore, as described earlier, the absorption peak due to ¹³CO₂ may possibly overlap the absorption peak due to ¹⁴CO₂ in an absorption spectrum.

If the pressure of the measurement-target gas is changed from the optimum pressure condition for the measurement of ¹⁴CO₂, the degrees of absorption by ¹²CO₂, ¹³CO₂ and ¹⁴CO₂ will also change. Depending on the pressure or the wavelength to be observed, the absorption by ¹⁴CO₂ may be as low as practically negligible. Even if there is still a non-negligible amount of absorption by ¹⁴CO₂, the ratio of the absorption by ¹⁴CO₂ will be dramatically lower than under the optimum pressure condition. The second pressure in the present invention is, for example, a pressure at which the absorption by the target component, ¹⁴CO₂, is negligible or sufficiently low as compared to the absorption by ¹²CO₂, ¹³CO₂ and other non-targeted components which are present in the measurement-target gas.

For example, if the absorption by the target component ¹⁴CO₂ under the second pressure is as low as negligible as compared to the absorption by ¹²CO₂, ¹³CO₂ and other non-targeted components, the measurement in the second measurement step will yield a result (ring-down rate or ring-down time) which only reflects the absorption by the non-targeted components. That is to say, this measurement result is substantially free from the influence of the absorption by the target component, and therefore, the absorption observed in that result can be considered as the background. Accordingly, in the calculation step, a calculation for removing or reducing the influence of the background is performed using measured results obtained by two CRDS measurements performed at different pressures of the measurement-target gas, to calculate the concentration of the target component.

In the gas measurement method and gas measurement device according to the present invention, the wavelength of the laser light used in the measurement in the second measurement step may be equal to the wavelength of the laser light used in the measurement in the first measurement step. In other words, even when the same wavelength of laser light is used for the measurement by CRDS in both of the first and second measurement steps, the signal corresponding to the absorption by a low-concentration component, such as ¹⁴CO₂, can be separated from an overlapping signal corresponding to the absorption by a high-concentration component, such as ¹²CO₂ or ¹³CO₂, and the absorption coefficient and concentration of the low-concentration component can be calculated.

If the wavelength of the laser light is varied over a wide range in order to accurately perform spectrum fitting in a gas measurement device employing the CRDS method, a laser source capable of sweeping a wide range of wavelengths is naturally required. Additionally, an optical resonator having high-reflection mirrors compatible with that wavelength-swept range is also required. By comparison, the present invention only needs to perform two measurements without changing the wavelength of the laser light. It is normally unnecessary to use a laser light capable of sweeping a wide range of wavelengths or switching the wavelength over a wide range required for ensuring the accuracy of the spectrum fitting, or an optical resonator having high-reflection mirrors compatible with that range of wavelengths.

Changing the pressure of the measurement-target gas to perform the measurement of the background also has the following advantage:

As noted earlier, one of the most important features of the CRDS is its high level of detection sensitivity. Therefore, in many cases, the concentration of the target component is considerably low, which is often close to the lower limit of the detection. In such a case, the amounts of absorption by other components around the absorption peak of the target component are also comparatively small, and a measured absorption coefficient to be used for estimating the spectral waveform of the background by the fitting operation may not be sufficiently accurate from the start. In that case, even when there is no absorption peak due to non-targeted components as shown in FIG. 11A, the spectral waveform of the background will be inaccurate. Using this spectral waveform will lower the accuracy of the background removal and cause the concentration of the target component to be less accurate.

By comparison, for example, in the case where the target component is ¹⁴CO₂, if the pressure of the measurement-target gas is increased to a higher level than the optimum pressure for the measurement of ¹⁴CO₂, the absorption by ¹²CO₂ or ¹³CO₂ will dramatically increase as compared to the absorption by ¹⁴CO₂. Therefore, the overall background level will increase. This increases the accuracy of the result of the measurement of the background, making it possible to accurately perform the background removal and more accurately determine the concentration of the target component.

Each of the first and second pressures can be determined beforehand by calculations or experiments depending on the target component.

As described earlier, even when the absorption by the target component under the second pressure is non-negligible as compared to the absorption by the non-targeted components in the measurement-target gas, the absorption coefficient and concentration of the target component can be determined by using the fact that the ratio of the absorption by the plurality of components changes depending on the pressure.

Thus, as one mode of the gas measurement method according to the present invention:

the second measurement step may be for performing the measurement by cavity ring-down absorption spectroscopy for the wavelength of the absorption peak of the target component; and

the calculation step may be for preparing a system of equations based on the measurement result of the first measurement step and the measurement result of the second measurement step, and to solve the system of equations to calculate the concentration of the target component from or in which an influence of the non-targeted components in the measurement-target gas is removed or reduced.

Additionally, as one mode of the gas measurement device according to the present invention:

the controller may be configured to perform the measurement by cavity ring-down absorption spectroscopy for the wavelength of the absorption peak of the target component in the measurement under the second pressure; and

the calculation processor may be configured to prepare a system of equations based on a measurement result obtained under the first pressure and a measurement result obtained under the second pressure, and to solve the system of equations to calculate the concentration of the target component from or in which an influence of the non-targeted components in the measurement-target gas is removed or reduced.

In these modes of the present invention, since the influence of the absorption by the target component is also included in the background, a system of equations in which the concentration or absorption coefficient of the target component as well as the concentration or absorption coefficient of the non-targeted components are included as unknown values is solved to calculate the concentration of only the target component. By this method, even when the pressure of the measurement-target gas cannot be changed to a level at which the influence of the absorption by the target component is completely eliminated, the influence of the background can be appropriately removed to obtain the absorption coefficient and concentration of the target component.

The wavelength of the laser light used in the measurement in the second measurement step does not always need to be equal to that of the laser light used in the measurement in the first measurement step.

Thus, as another mode of the gas measurement method according to the present invention, the second measurement step may be for performing the measurement by cavity ring-down absorption spectroscopy for a wavelength at which an influence of the absorption by the target component is negligible, the wavelength being different from the wavelength of the absorption peak of the target component; and

the calculation step may be for estimating the concentration of non-targeted components in the measurement-target gas under the second pressure based on the measurement result of the second measurement step, then estimate, from that concentration, a contribution of the absorption by the non-targeted components to an absorption coefficient determined from the measurement result of the first measurement step, and perform a calculation which removes an influence of the absorption by the non-targeted components.

Additionally, as another mode of the gas measurement device according to the present invention:

the controller may be configured to perform, in the measurement under the second pressure, the measurement by cavity ring-down absorption spectroscopy for a wavelength at which an influence of the absorption by the target component is negligible, the wavelength being different from the wavelength of the absorption peak of the target component; and

the calculation processor may be configured to estimate, based on a measurement result obtained under the second pressure, the concentration of non-targeted components in the measurement-target gas under the second pressure, then estimate, from that concentration, a contribution of the absorption by the non-targeted components to an absorption coefficient determined from a measurement result obtained under the first pressure, and perform a calculation which removes an influence of the absorption by the non-targeted components.

In the present case, the wavelength of the laser light used in the measurement in the second measurement step may be an appropriate wavelength at which it has been previously confirmed that the influence of the absorption by the target component is negligible. Although these modes require the wavelength of the laser light used for the measurement to be switched between the first and second measurement steps, a benefit exists in that the measurement result obtained by the measurement in the second measurement step is practically free from the absorption by the target component. Therefore, the measurement result can be considered as the absorption of only the non-targeted components, i.e., the background. As compared to the previously described case of solving a system of equations, the removal of the background is easier to achieve. Even when the pressure of the measurement-target gas cannot be changed to a level at which the influence of the absorption by the target component is completely eliminated, the background-removing process can be performed in a comparatively easy manner.

Although the previously described modes require the switching of the wavelength of the laser light, it is normally unnecessary to measure the absorption coefficient at a remote wavelength, as in the case of using another absorption peak of the target component; what is necessary is to change the wavelength within a narrow wavelength range. Accordingly, even light sources and mirrors which are commonly used in CRDS devices can be used without any problem.

In the gas measurement device according to the present invention, the pressure regulator may be configured to regulate the pressure of the measurement-target gas in the measurement cell to the first pressure by compulsorily discharging a portion of the measurement-target gas from the measurement cell to the outside, starting from a state in which the measurement cell contains the measurement-target gas under the second pressure.

Specifically, the pressure regulator may include: an on/off valve provided in a gas introduction tube connected to the measurement cell; an on/off valve provided in a gas discharge tube connected to the measurement cell; a vacuum pump configured to discharge the measurement-target gas from the measurement cell to the outside through the gas discharge tube; a pressure detector configured to detect the pressure of the gas in the measurement cell; and a pressure controller configured to control an on/off operation of the on/off valves as well as an operation of the vacuum pump while monitoring the pressure with the pressure sensor.

Alternatively, in the gas measurement device according to the present invention, the pressure regulator may be configured to regulate the pressure of the measurement-target gas in the measurement cell to the second pressure by additionally supplying the measurement cell with the measurement-target gas which remains unsupplied, starting from a state in which the measurement cell is filled with the measurement-target gas supplied beforehand and contains the measurement-target gas under the first pressure.

Specifically, the pressure regulator may include: an on/off valve provided in a gas introduction tube connected to the measurement cell; an on/off valve provided in a gas discharge tube connected to the measurement cell; a supply pump configured to supply the measurement-target gas into the measurement cell through the gas introduction tube; a pressure detector configured to detect the pressure of the gas in the measurement cell; and a pressure controller configured to control an on/off operation of the on/off valves as well as an operation of the supply pump while monitoring the pressure with the pressure sensor.

According to those configurations, the pressure of the measurement-target gas in the measurement cell can be easily regulated to a desired value.

### Advantageous Effects of Invention

According to the present invention, the background due to the absorption by non-targeted components in a measurement-target gas can be removed with a high level of accuracy by two measurements performed for the measurement-target gas, and an accurate concentration of the target component can be obtained. This eliminates the necessity of repeating a measurement a considerable number of times for estimating the spectrum of the background, so that the measurement time can be shortened, and the measurement throughput can be improved. The shortened measurement time is also advantageous in that the concentration measurement can be accurately performed even when the target component is comparatively unstable, as in the case of a radioactive isotope having a short half-life.

Furthermore, according to the present invention, due to the shortened measurement time, the influence of a change in measurement conditions which occurs in the middle of the measurement can be suppressed to the minimum or close to the minimum, where the change in measurement conditions may be a decrease in the effective reflectance of a mirror due to the adsorption of a portion of the measurement gas to the mirror, a change in effective reflectance or resonator length due to a slight change in the position of a mirror or due to an infinitesimal shift of the incident position of the incoming light, or a change in the resonator length or other related dimensions due to a thermal expansion of the optical resonator caused by a slight change in temperature.

Furthermore, according to the present invention, even when the absorption peak of the target component and that of another component overlap each other, the background can be accurately estimated and removed so that the absorption coefficient and concentration of the target component can be determined with a high level of accuracy.

### BRIEF DESCRIPTION OF DRAWINGS

FIG. 1 is a configuration diagram of the main components of a CRDS device as one embodiment of the present invention.

FIGS. 2A and 2B are graphs showing calculation results of the characteristics of the absorption by CO₂ isotope gases at a pressure of 1013.25 Pa.

FIGS. 3A-3C are graphs showing calculation results of the characteristics of the absorption by CO₂ isotope gases at a pressure of 10132.5 Pa.

FIG. 4 is a flowchart showing one example of the procedure of the measurement and processing in the case of determining the concentration of a target component in the CRDS device according to the present embodiment.

FIG. 5 is a flowchart showing another example of the procedure of the measurement and processing in the case of determining the concentration of a target component in the CRDS device according to the present embodiment.

FIGS. 6A and 6B are diagrams showing calculation results of the characteristics of the absorption by H₂O isotope gases at a pressure of 1013.25 Pa.

FIGS. 7A and 7B are diagrams showing calculation results of the characteristics of the absorption by H₂O isotope gases at a pressure of 101325 Pa.

FIG. 8 is a schematic configuration diagram of a commonly used CRDS device.

FIG. 9 is a schematic diagram showing a relationship between mode frequency and laser oscillation frequency in an optical resonator.

FIG. 10 is a schematic diagram of an absorption spectrum of a measurement-target gas around a peak wavelength of an absorption line of ¹⁴CO₂.

FIG. 11A is a graph showing a calculation result of an absorption spectrum of CO₂ isotope gases, and FIG. 11B is a chart showing a calculation result of the degrees of contribution of the absorption by ¹²CO₂, ¹³CO₂ and ¹⁴CO₂ at the position of the absorption peak of ¹⁴CO₂.

FIG. 12A is a graph showing a calculation result of an absorption spectrum of CO₂ isotope gases under the condition that the pressure of the measurement-target gas is increased, and FIG. 12B is a chart showing a calculation result of the degrees of contribution of the absorption by ¹²CO₂, ¹³CO₂ and ¹⁴CO₂ at the position of the absorption peak of ¹⁴CO₂.

### DESCRIPTION OF EMBODIMENTS

A CRDS device as one embodiment of the gas measurement device according to the present invention, and a gas measurement method using the same device, are hereinafter described with reference to the attached drawings.

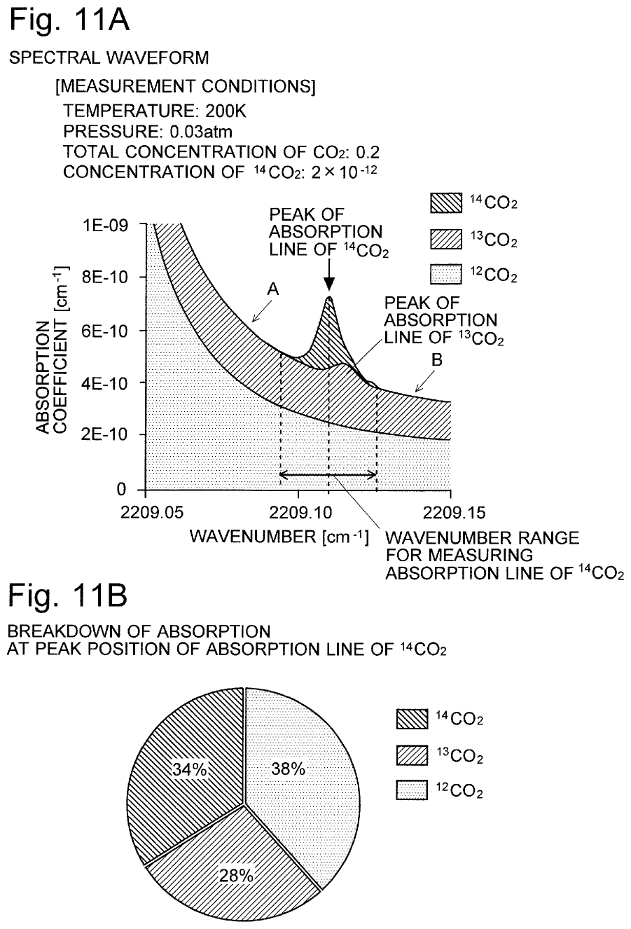

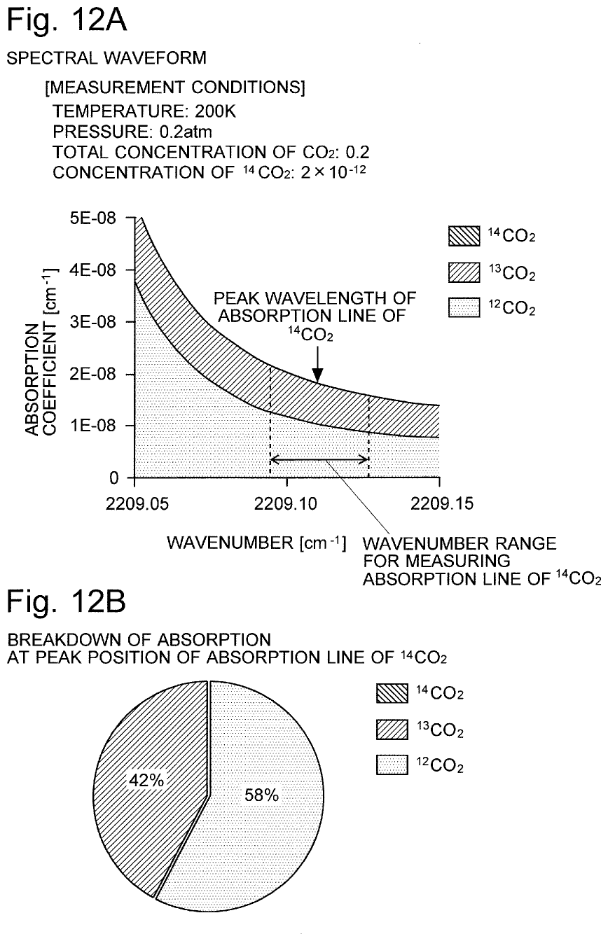

Initially, using FIGS. 11A-12B, the previously described problem to be solved by the present invention will be summarized, and the principle of the background removal in the present invention will be described.

FIGS. 11A and 12A each show a calculation result of an absorption spectrum of ¹²CO₂, ¹³CO₂ and ¹⁴CO₂ around the position of an absorption peak of ¹⁴CO₂. The only difference between FIGS. 11A and 12A is the assumed pressure of the measurement-target gas. Specifically, when the pressure is relatively low (in the present case, 0.03 atm), the absorption peak of ¹⁴CO₂ can be clearly observed, whereas the absorption peak of ¹³CO₂ whose base portion overlaps the absorption peak of ¹⁴CO₂ cannot be recognized in an isolated form. Therefore, spectrum fitting for the absorption by ¹³CO₂ cannot be correctly performed, and it is difficult to accurately estimate the background (i.e., the baseline of the peak).

A gas molecule normally has a plurality of absorption peaks corresponding to its rotation, translation and vibration. Even when signals overlap each other in an absorption peak at one wavelength in the previously described manner, the absorption coefficient may possibly be determined by using another absorption peak at another wavelength. However, the light source and mirrors used in a CRDS device each have a limited wavelength range within which it can work properly. It is often the case that the device cannot deal with the wavelengths of a plurality of absorption peaks of a target component in a measurement-target gas. There has practically been no effective method for measuring the absorption coefficient in such cases.

In a conventional CRDS measurement, it is assumed that the pressure and temperature of the measurement-target gas are constantly maintained. When the concentration of ¹⁴CO₂ needs to be determined, it is normal to set the pressure so that the absorption peak of ¹⁴CO₂ will be as high as possible, as shown in FIG. 11A. On the other hand, when the pressure of the measurement-target gas is increased to an appropriate level, the absorption peak of ¹³CO₂ disappears almost completely along with that of ¹⁴CO₂, as shown in FIG. 12A. However, this does not mean that the absorption by ¹³CO₂ has disappeared; it merely means that the absorption peak has disappeared, and the absorption itself is still present, as shown in FIGS. 12A and 12B. There is also the absorption by ¹²CO₂ having a higher concentration. That is to say, when the pressure of the measurement-target gas is increased as shown in FIG. 12A, the absorption at the position of the absorption peak of ¹⁴CO₂ can be considered to be due to the components other than the target component (¹⁴CO₂), i.e., due to the background only. It should be noted that increasing the pressure increases the amount of absorption, so that the absorption coefficient itself has also significantly increased.

The CRDS measurement with the pressure of the measurement-target gas increased in the previously described manner yields a result (ring-down rate or ring-down time) which reflects the absorption by the non-targeted components. Based on the absorption coefficient calculated from this result, the absolute concentration of the non-targeted components can be calculated. From this absolute concentration, the spectrum of the baseline at the position of the absorption peak of ¹⁴CO₂ under the condition that the pressure of the measurement-target gas is relatively low as shown in FIG. 11A can be estimated. By subtracting this baseline from the absorption coefficient determined from a measurement result obtained by performing the CRDS measurement with a relatively low pressure of the measurement-target gas, the absorption coefficient of pure ¹⁴CO₂ can be determined. From this absorption coefficient, the absolute concentration of only the target component, ¹⁴CO₂, can be calculated.

Depending on the pressure of the measurement-target gas, the degree of absorption of ¹⁴CO₂ may be non-negligible. Even in that case, the absolute concentration of only the target component ¹⁴CO₂ can be calculated by solving a system of equations, as will be described later. As another possibility, the wavelength of the laser light used in the measurement may be varied in addition to the pressure of the measurement-target gas, to acquire a measurement result which reflects the baseline spectrum that is free from the influence of the absorption by the target component, and use that measurement result to determine the absorption coefficient of pure ¹⁴CO₂. This will also be described later.

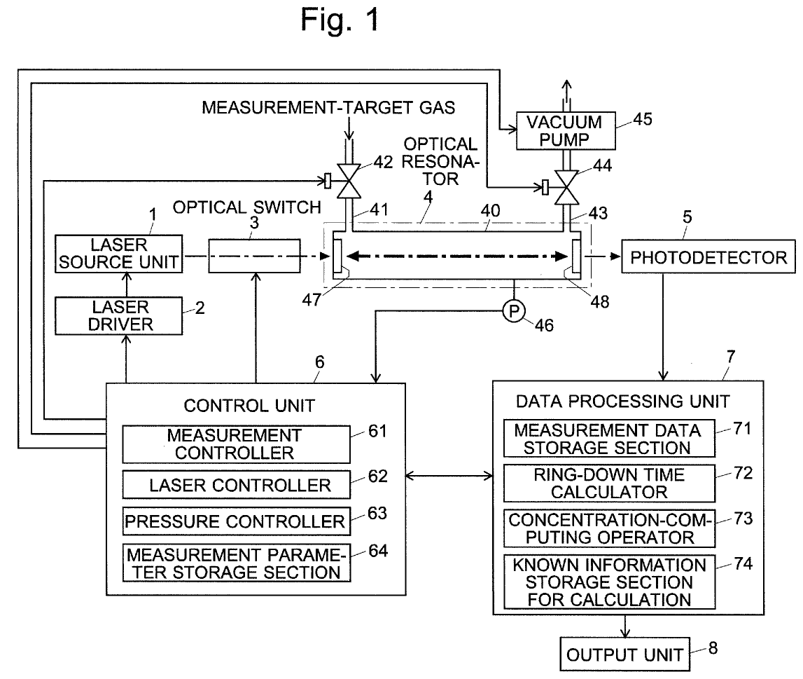

One example of the CRDS device employing the previously described measurement principle is hereinafter described. FIG. 1 is a configuration diagram of the main components of the CRDS device according to the present embodiment.

The CRDS device according to the present embodiment has, as its measurement system, a laser source unit 1, laser driver 2, optical switch 3, optical resonator 4 and photodetector 5. The optical resonator 4 includes a substantially cylindrical measurement cell 40 configured to contain a sample gas as a measurement-target gas, as well as a pair of high-reflection mirrors 47 and 48 arranged at both ends of the measurement cell 40 and facing each other. A gas introduction tube 41 and gas discharge tube 43 are connected to the measurement cell 40. The gas introduction tube 41 has an introduction valve 42, while the gas discharge tube 43 has a discharge valve 44 and a vacuum pump 45. The measurement cell 40 is equipped with a pressure sensor 46 for detecting the pressure of the gas contained in the cell 40.

A control unit 6 is responsible for controlling the laser driver 2 and other related components in order to perform measurements and data processing as will be described later. This unit includes, as its functional blocks, a measurement controller 61, laser controller 62, pressure controller 63, measurement parameter storage section 64 and other related sections. In the measurement parameter storage section 64, measurement parameters, including the wavenumber (or wavelength) of the laser light and the pressure, are previously stored, being related to the kind of component to be subjected to the measurement or other types of information. A data processing unit 7, which receives detection signals from the photodetector 5, includes a measured data storage section 71, ring-down time calculator 72, concentration-computing operator 73, known information storage section for calculation 74, and other related sections as its functional blocks. The measured data storage section 71 includes an analogue-to-digital converter configured to digitize analogue detection signals. An output unit 8 connected to the data processing unit 7 is, for example, a display monitor.

A specific example of the operation in the CRDS device according to the present embodiment is hereinafter described on the assumption that the measurement-target gas is CO₂, and the target component is ¹⁴CO₂, which is one of the isotopes of CO₂. Concentration measurements of ¹⁴CO₂, which contains radioactive carbon ¹⁴C, have been widely used in various areas.

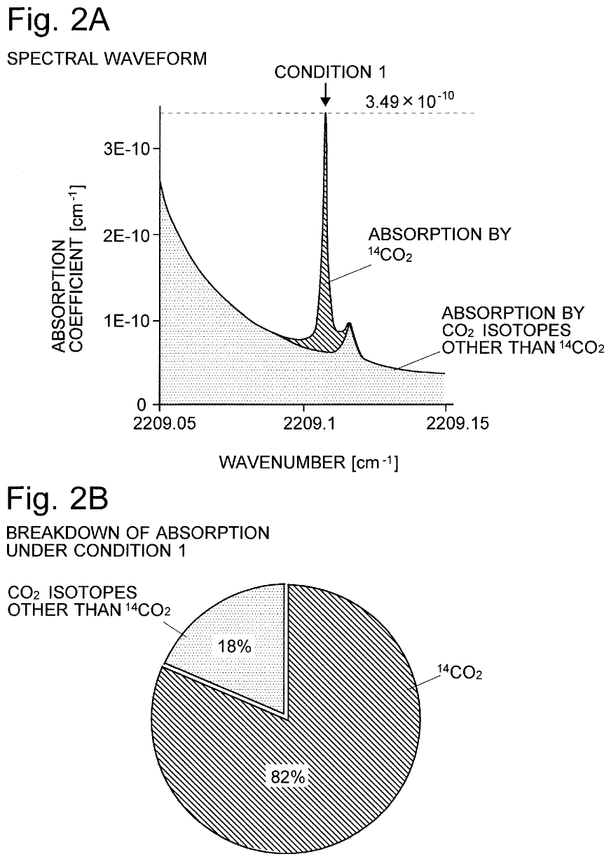

FIG. 2A is a graph showing the result of a calculation of the absorption spectrum by CRDS for CO₂ isotope gases at a pressure of 1013.25 Pa (=0.01 atm). The horizontal axis represents the wavenumber of light, and the vertical axis represents absorption coefficient. FIG. 2B is a pie chart showing a breakdown of the cause of the absorption at the wavenumber of light labelled “Condition 1” (the wavenumber of the absorption peak of ¹⁴CO₂) in FIG. 2A. For the calculation, it was assumed that the temperature was 200 K, the ¹⁴CO₂ concentration was 2×10⁻¹², and the CO₂ concentration exclusive of ¹⁴CO₂ was 0.2. It was also assumed that the CO₂ isotopes, other than ¹⁴CO₂, contained in the measurement-target gas were introduced into the measurement cell 40 with their natural isotope abundance ratio.

As written in FIG. 2A, the calculated absorption coefficient at the wavenumber of the absorption peak of ¹⁴CO₂ at the aforementioned pressure was 3.49×10⁻¹⁰. As can be seen in FIG. 2B, approximately 82% of the absorption of light in the present case is due to ¹⁴CO₂, while the remaining portion, approximately 18%, is due to the other kinds of CO₂ isotopes (¹²CO₂ and ¹³CO₂). Accordingly, in order to calculate an accurate concentration of ¹⁴CO₂, it is necessary to subtract the baseline due to the absorption by CO₂ exclusive of ¹⁴CO₂. The spectrum of this baseline can be determined if the concentration of CO₂ exclusive of ¹⁴CO₂ is known. This information can be easily obtained if the absorption coefficient due to CO₂ exclusive of ¹⁴CO₂ can be determined from the measured data. However, a problem exists in that accurate data which reflects the amount of absorption by CO₂ exclusive of ¹⁴CO₂ cannot be obtained in the present situation since the amount of absorption in question is considerably low under measurement conditions that are optimized for ¹⁴CO₂ so that a sharp absorption peak of ¹⁴CO₂ can be observed as shown in FIG. 2A. In order to overcome this problem, in the present invention, measurements are performed at different pressures of the measurement-target gas, and the results of those measurements are used to determine the spectrum of the baseline due to the absorption by CO₂ exclusive of ¹⁴CO₂.

In a common type of CRDS, the measurement for a measurement-target gas is performed with the pressure constantly maintained throughout the measurement. As is commonly known, the absorption coefficient of a component in a gas depends on temperature, pressure, wavelength of light and other related factors. Accordingly, in common cases, the pressure for a measurement of the absorption by ¹⁴CO₂ is set at a level that satisfies, for example, the condition that the difference between the absorption coefficient of the target component ¹⁴CO₂ and that of CO₂ exclusive of ¹⁴CO₂ becomes as large as possible at the wavenumber of an absorption peak due to ¹⁴CO₂, because such a pressure condition is likely to yield the best signal-to-noise ratio for the observation of the absorption peak due to ¹⁴CO₂. Increasing the pressure of the measurement-target gas to be higher than the aforementioned level actually causes a dramatic increase in the heights of the absorption peaks of ¹²CO₂ and ¹³CO₂ at the wavenumbers of their respective absorption peaks, along with an increase in their peak widths. Consequently, the background level becomes considerably high, which lowers the signal-to-noise ratio of the absorption peak due to ¹⁴CO₂.

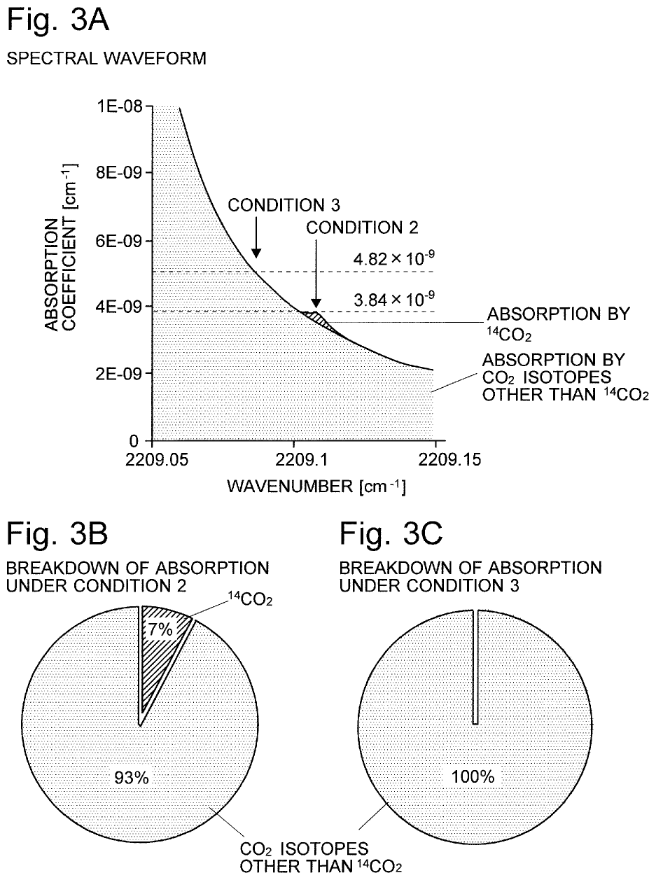

FIG. 3A is a graph showing the result of a calculation of the absorption spectrum by CRDS for CO₂ isotope gases at a pressure of 10132.5 Pa (=0.1 atm), which is 10 times the pressure used in the case of FIG. 2A. FIG. 3B is a pie chart showing a breakdown of the cause of the absorption at a wavenumber labelled “Condition 2” in FIG. 3A (which is the same as the wavenumber under the aforementioned “Condition 1”). FIG. 3C is a pie chart showing a breakdown of the cause of the absorption at a wavenumber labelled “Condition 3” in FIG. 3A (which is an appropriate wavenumber smaller than “Condition 1”). Calculation conditions other than the pressure are identical to those in the case of FIGS. 2A and 2B.

The wavenumber of the absorption peak due to ¹²CO₂ is located beyond the left end of the graph shown in FIG. 3A. The height and peak width of that absorption peak rapidly increases with an increase in pressure. Although the height of the absorption peak due to ¹⁴CO₂ in the present situation is larger than ten times the height of the same absorption peak in the graph shown in FIG. 2A, the absorption peak is almost entirely buried in the tailing portion of the absorption peak due to ¹²CO₂. This demonstrates that increasing the pressure of the measurement-target gas considerably increases the overall level of the background, making it easier to detect the absorption by CO₂ exclusive of ¹⁴CO₂, or improving the detection accuracy for that absorption. Accordingly, in the present invention, in addition to a CRDS measurement under a pressure condition in which the ratio of the absorption by the target component (¹⁴CO₂) is relatively large, another CRDS measurement is performed for the same measurement-target gas under a pressure condition under which the level of the background becomes higher as just described.

As shown in FIGS. 3B and 3C, the absorption by ¹⁴CO₂ still forms approximately 7% of the entire absorption at the wavenumber of Condition 2, whereas the percentage of the absorption by ¹⁴CO₂ is zero at the wavenumber of Condition 3. The measurement which must be performed at a relatively high pressure for determining the background only needs to be performed under any one of the Conditions 2 and 3, although the method for processing the measurement result changes depending on which condition is used for the measurement. It should be noted that the wavenumber of Condition 3 is not limited to the position show in FIG. 3A. Any wavenumber that falls within a controllable range of the wavenumber of the laser light may be appropriately selected as long as the absorption peak of ¹⁴CO₂ is buried in the background, and the level of the background is high (i.e., as long as the wavenumber is located on the left side of the position of the absorption peak of ¹⁴CO₂ in FIG. 3A).

[Condition 3: When Absorption by ¹⁴CO₂ is Negligible]

At the wavenumber of Condition 3, the background due to the absorption by ¹²CO₂ and ¹³CO₂ is at such a high level that the absorption by ¹⁴CO₂ is negligible. Therefore, there is practically no influence of the absorption by ¹⁴CO₂ appearing in the result of the CRDS measurement. Accordingly, it is possible to calculate the absorption coefficient of the CO₂ isotopes other than ¹⁴CO₂ based on the ring-down time determined from the thereby obtained measurement data, and to calculate, from that absorption coefficient, the concentration of the CO₂ isotopes other than ¹⁴CO₂ at the relatively high pressure. By using the calculated concentration of the CO₂ isotopes other than ¹⁴CO₂, the absorption coefficient corresponding to the background at the wavenumber of Condition 1 at the relatively low pressure with a strong absorption by ¹⁴CO₂ can be calculated. Accordingly, by subtracting the absorption coefficient corresponding to the background from the absorption coefficient determined from the measured result obtained under Condition 1, the absorption coefficient of only ¹⁴CO₂ can be determined, and from this absorption coefficient, the concentration of only the target component ¹⁴CO₂ can be calculated.

[Condition 2: When Absorption by ¹⁴CO₂ is Non-Negligible]

At the wavenumber of Condition 2, the absorption by ¹⁴CO₂ forms approximately 7% of the entire absorption, and is therefore non-negligible. In this case, a system of equations in which the concentration of ¹⁴CO₂ and that of the CO₂ isotopes other than ¹⁴CO₂ are included as unknowns is prepared based on the measured result at the wavenumber of the absorption peak of ¹⁴CO₂ obtained at a relatively low pressure and the measured result at the wavenumber of Condition 2 obtained at a relatively high pressure. By solving the system of equations, the concentration of ¹⁴CO₂ and that of CO₂ isotopes other than ¹⁴CO₂ can be calculated. Alternatively, a system of equations in which the absorption coefficient of ¹⁴CO₂ and that of the CO₂ isotopes other than ¹⁴CO₂ are included as unknowns may be prepared and solved.

In any of these cases, the concentration of ¹⁴CO₂ calculated in the previously described manner is free from the influence of the absorption by the CO₂ isotopes other than ¹⁴CO₂, or the influence is practically negligible. Therefore, the concentration of ¹⁴CO₂ in the measurement-target gas can be determined with a high level of accuracy. The pressure condition and the wavenumber of the used laser light for each of the measurements can be appropriately determined beforehand according to the kind of component to be subjected to the measurement or other related factors.

In the previously described methods, the concentration of the CO₂ isotopes other than ¹⁴CO₂ is determined without discriminating between ¹²CO₂ and ¹³CO₂. When it is necessary to individually determine the concentrations of ¹²CO₂, ¹³CO₂ and ¹⁴CO₂, a measurement can be performed for each isotope gas under a different pressure condition which significantly increases the percentage of the absorption of the isotope gas in question, and the concentrations of the individual isotope gases can be calculated from the results of the three measurements.

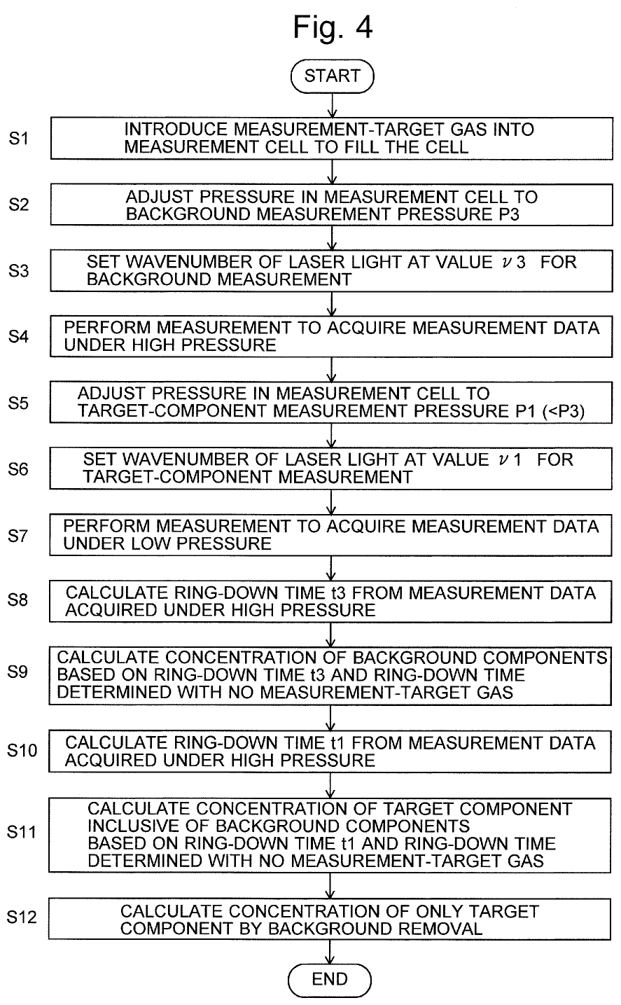

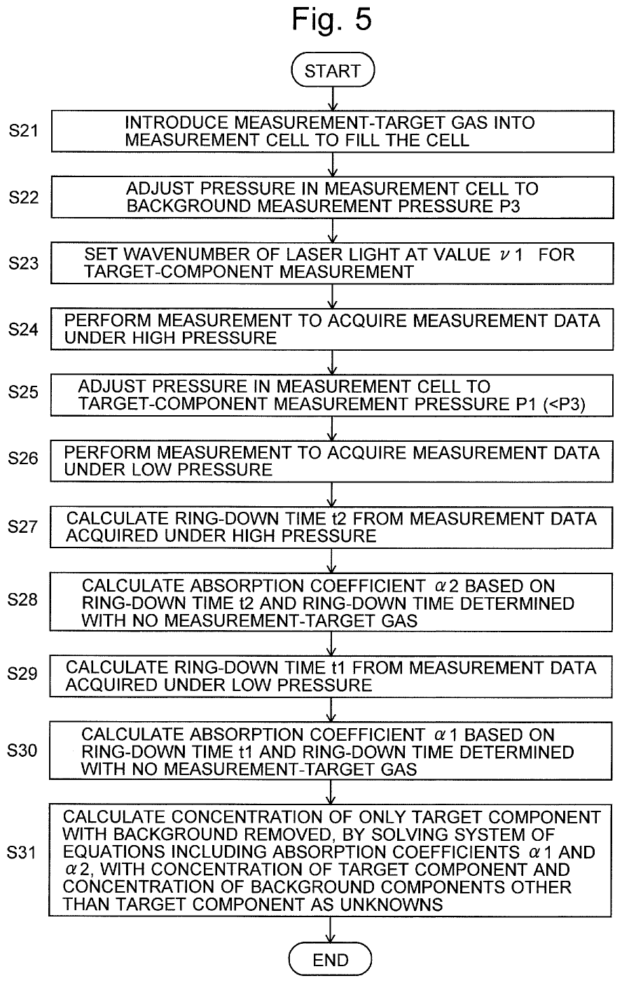

Flowcharts of the operations for measuring the concentration of the target component (¹⁴CO₂) in the measurement-target gas in the CRDS device according to the present embodiment are shown in FIGS. 4 and 5.

FIG. 4 is a flowchart showing one example of the procedure of the measurement and processing in the case of removing the background using a measured result obtained under Condition 3. It is assumed that the ring-down time at each pressure under the condition that the measurement-target gas containing CO₂ is not present in the measurement cell 40 has been previously measured and is stored in the known information storage section for calculation 74. It is also assumed that prior information to be used for computing concentrations, such as the absorption cross section of the target component, is also stored in the known information storage section for calculation 74.

Initially, the pressure controller 63 in the control unit 6 opens the introduction valve 42, with the discharge valve 44 closed, to introduce a measurement-target gas into the measurement cell 40. When the pressure detected with the pressure sensor 46 has reached a predetermined level, the pressure controller 63 closes the introduction valve 42 to fill the measurement cell 40 with the measurement-target gas (Step S1). Subsequently, the pressure controller 63 opens the discharge valve 44 and energizes the vacuum pump 45, whereby the measurement-target gas in the measurement cell 40 begins to be discharged through the gas discharge tube 43. When the pressure detected with the pressure sensor 46 has decreased to a predetermined background (BG) measurement pressure P3 stored in the measurement parameter storage section 64, the pressure controller 63 closes the discharge valve 44 (Step S2). Consequently, the measurement cell 40 contains the measurement-target gas at pressure P3.

The laser controller 62 operates the laser source unit 1 through the laser driver 2 so that the wavenumber of the laser light will be a predetermined value v3 for the background (BG) measurement (Step S3). The measurement controller 61 performs the measurement under the condition of laser-light wavenumber v3 and pressure P3. Specifically, the measurement-target gas in the measurement cell 40 is irradiated with laser light, and the laser light is blocked at a predetermined timing with the optical switch 3. The data acquired with the photodetector 5 is collected from immediately before the blocking of the laser light until a predetermined period of time elapses (Step S4). The measurement data acquired at high pressure with the photodetector 5 in this step is temporarily stored in the measured data storage section 71. The measurement data acquired in this step is a set of data which contains, as a piece of information, a ring-down time t3 under Condition 3.

Subsequently, the pressure controller 63 opens the discharge valve 44 once more and energizes the vacuum pump 45, whereby the measurement-target gas in the measurement cell 40 begins to be discharged through the gas discharge tube 43 to the outside. When the pressure detected with the pressure sensor 46 has decreased to a target-component measurement pressure P1 stored in the known information storage section for calculation 74, the pressure controller 63 closes the discharge valve 44 (Step S5). Consequently, the measurement cell 40 contains the measurement-target gas at pressure P1, which is lower than P3.

Meanwhile, the laser controller 62 operates the laser source unit 1 through the laser driver 2 so that the wavenumber of the laser light will be a predetermined value v1 for the target-component measurement (Step S6). The measurement controller 61 performs the measurement under the condition of laser-light wavenumber v1 and pressure P1 to acquire measurement data over a predetermined period of time, as in Step S4 (Step S7). The measurement data sequentially acquired with the photodetector 5 in this step is also temporarily stored in the measured data storage section 71. This measurement data acquired at low pressure is a set of data which contains, as a piece of information, a ring-down time t1 under Condition 1 shown in FIG. 2A.

Strictly speaking, the measurement-target gas subjected to the measurement in Step S7 is not perfectly identical to the measurement-target gas in Step S4 since a portion of the measurement-target gas in the measurement cell 40 is discharged to the outside in Step S5 to decrease the pressure. However, since the distribution of the components in the measurement-target gas within the measurement cell 40 can be considered as uniform, the measurement-target gases subjected to the measurements in Steps S4 and S7 can be considered to be identical and merely different from each other in pressure.

In the data processing unit 7, the ring-down time calculator 72 calculates the ring-down time t3 based on the measurement data which has been acquired at high pressure and stored in the measured data storage section 71 (Step S8). Based on this calculated result as well as the ring-down time determined under Condition 3 with no measurement-target gas and stored in the known information storage section for calculation 74, the concentration-computing operator 73 calculates the absorption coefficient and determines the concentration from that absorption coefficient (Step S9). Those calculations are performed in the same manner as in a conventional method, in which the aforementioned equations (1) and (2) can be used, for example. In the present case, since the influence of the absorption by ¹⁴CO₂ is negligible, the value obtained in Step S9 is the concentration of the CO₂ isotope gas exclusive of ¹⁴CO₂ in the measurement-target gas.

Subsequently, the ring-down time calculator 72 calculates the ring-down time t1 based on the measurement data which has been acquired at low pressure and stored in the measured data storage section 71 (Step S10). Based on this calculated result as well as the ring-down time determined under Condition 1 with no measurement-target gas and stored in the known information storage section for calculation 74, the concentration-computing operator 73 calculates the absorption coefficient (Step S11). The value obtained by this calculation is the absorption coefficient of the CO₂ isotope gases inclusive of ¹⁴CO₂ in the measurement-target gas.

The concentration of the CO₂ isotope gas exclusive of ¹⁴CO₂ has already been obtained in Step S9. From this concentration, the concentration-computing operator 73 calculates the absorption coefficient due to the CO₂ isotope gas exclusive of ¹⁴CO₂ under the pressure and laser wavenumber of Condition 1. This absorption coefficient corresponds to the background. The concentration-computing operator 73 subtracts the absorption coefficient due to the CO₂ isotope gas exclusive of ¹⁴CO₂ from the absorption coefficient due to the CO₂ isotope gas inclusive of ¹⁴CO₂ to calculate the absorption coefficient due to ¹⁴CO₂ under Condition 1, and calculates the concentration of only ¹⁴CO₂ from this absorption coefficient (Step S12). The result is provided through the output unit 8.

As described to this point, the CRDS device according to the present embodiment can provide users with information of an accurate concentration of only ¹⁴CO₂ with the background removed.

Next, one example of the procedure of the measurement and processing in the case of removing the background using the measured result obtained under Condition 2 is described according to the flowchart shown in FIG. 5. Once again, it is assumed that a set of necessary information, including the ring-down time at each pressure under the condition that the measurement-target gas containing CO₂ is not present in the measurement cell 40 and the absorption cross section of the target component, is already stored in the known information storage section for calculation 74.

The processing in Steps S21 and S22 is identical to the processing in Steps S1 and S2. By those steps, the measurement cell 40 is filled with the measurement-target gas at pressure P3. The laser controller 62 operates the laser source unit 1 through the laser driver 2 so that the wavenumber of the laser light will be a predetermined value v1 for the target-component measurement (Step S23). The measurement controller 61 performs a measurement under the condition of laser-light wavenumber v1 and pressure P3 to acquire measurement data (Step S24). This measurement data acquired at high pressure is a set of data which contains, as a piece of information, a ring-down time t2 under Condition 2.

Next, the same processing as in Step S is performed in Step S25 to decrease the pressure of the measurement-target gas contained in the measurement cell 40 to the target-component measurement pressure P1. While maintaining the laser wavenumber at the value v1 for the target-component measurement, the measurement controller 61 performs a measurement under the condition of laser-light wavenumber v1 and pressure P1 to acquire measurement data (Step S26). This measurement data acquired at low pressure is a set of data which contains, as a piece of information, a ring-down time t1 under Condition 1, as in the example of FIG. 4.

In the data processing unit 7, the ring-down time calculator 72 calculates the ring-down time t2 based on the measured data acquired at high pressure and stored in the measured data storage section 71 (Step S27). Based on the calculated result and the ring-down time determined under Condition 2 with no measurement-target gas, the concentration-computing operator 73 calculates an absorption coefficient α2 (Step S28). Since the influence of the absorption by ¹⁴CO₂ is non-negligible in the present case, the value obtained in this step is the sum of the absorption coefficient due to ¹⁴CO₂ and the absorption coefficient due to the CO₂ isotope gas exclusive of ¹⁴CO₂ in the measurement-target gas under Condition 2.

Subsequently, the ring-down time calculator 72 calculates the ring-down time t1 based on the measurement data which has been acquired at low pressure and stored in the measured data storage section 71 (Step S29). This is identical to Step S10. Based on this calculated result as well as the ring-down time determined under Condition 1 with no measurement-target gas, the concentration-computing operator 73 calculates an absorption coefficient α1 (Step S30). The value obtained in this step is the sum of the absorption coefficient due to ¹⁴CO₂ and the absorption coefficient due to the CO₂ isotope gas exclusive of ¹⁴CO₂ in the measurement-target gas under Condition 1.

Both the concentration x of ¹⁴CO₂ and the concentration y of the CO₂ isotopes other than ¹⁴CO₂ are unknowns. Accordingly, a system of equations is prepared in which one equation expresses the relationship of the absorption coefficient α1 obtained by the measurement under Condition 1 and the concentrations x and y, while the other equation expresses the relationship of the absorption coefficient α2 obtained by the measurement under Condition 2 and the concentrations x and y. The concentration-computing operator 73 solves this system of equations to calculate the concentration of only ¹⁴CO₂ (Step S31).

As described to this point, an accurate concentration of only ¹⁴CO₂, with the background removed, can be calculated and presented to users.

In the previously described example, the absorption coefficient is determined from the ring-down time, and the concentration of ¹⁴CO₂ is subsequently calculated according to predetermined calculation formulae. Alternatively, the device may be configured so that the concentration of ¹⁴CO₂ can be derived by referring to a database instead of using calculation formulae. Specifically, for each of the measurement conditions (i.e., Conditions 1, 2 and 3), the values of the absorption coefficient observed for various combinations of the concentrations of ¹⁴CO₂ and other CO₂ isotopes are previously determined and compiled into a database. After an absorption coefficient has been determined from measured data, that absorption coefficient can be used as an input to conduct a database search and derive the corresponding concentration of ¹⁴CO₂ and that of the other CO₂ isotopes.

The previous description of the embodiment was concerned with the determination of the concentration of ¹⁴CO₂, which is one of the CO₂ isotopes. Understandably, the CRDS device according to the present embodiment is also available for an analysis of other kinds of components in a measurement-target gas. Hereinafter briefly described is a measurement of H₂O isotopes in a measurement-target gas as another application example. This type of measurement is comparable to the measurement of CO₂ isotopes in terms of utility value.

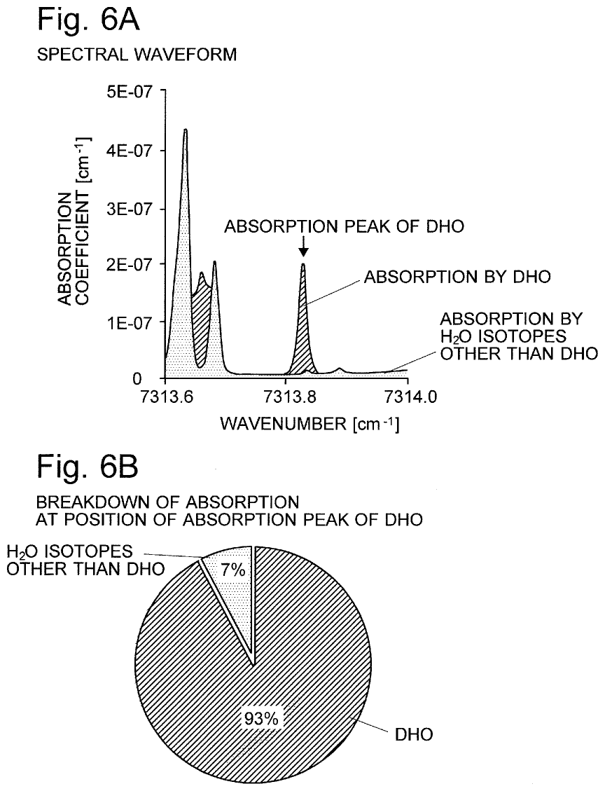

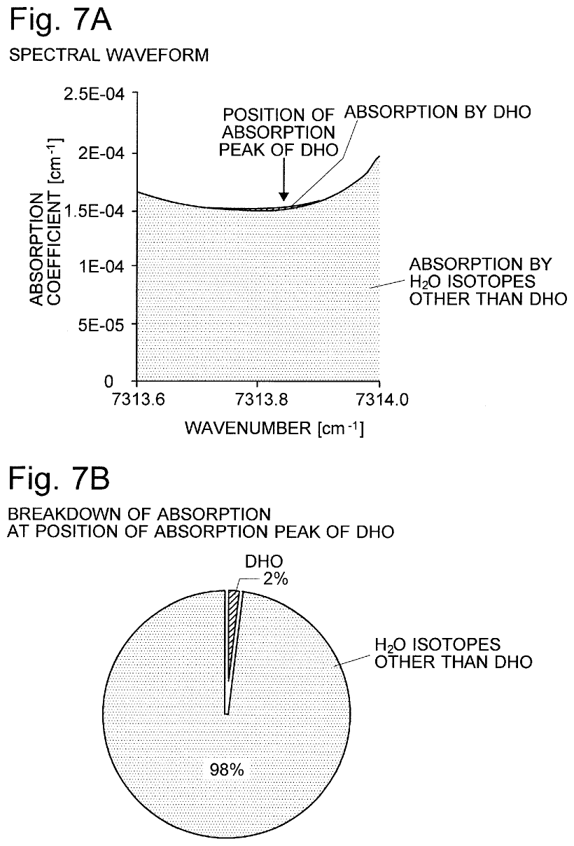

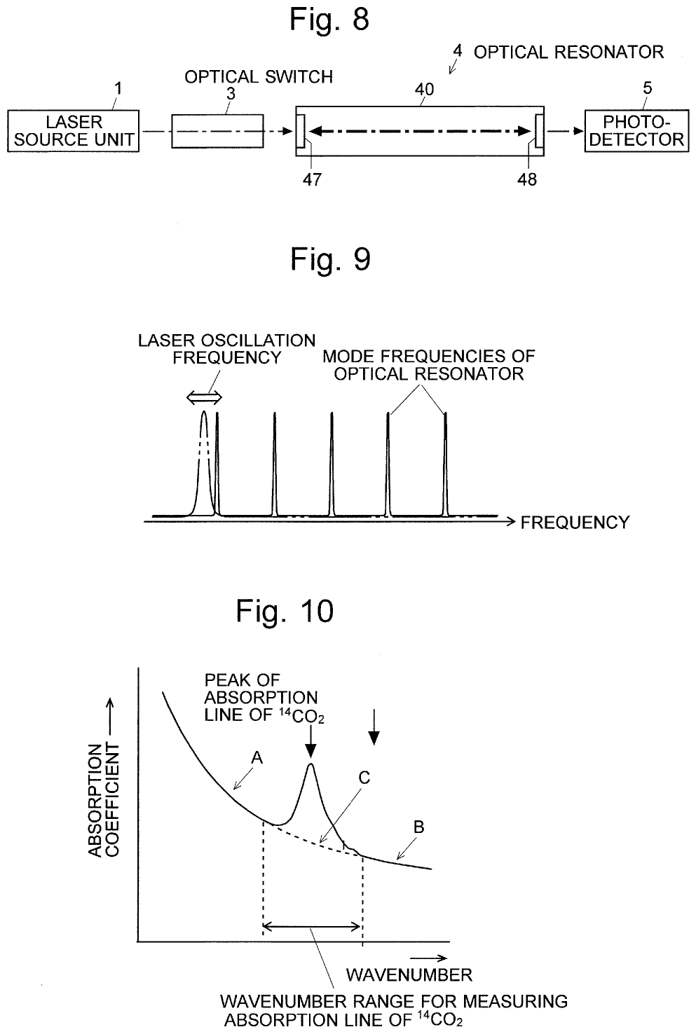

FIG. 6A is a graph showing the result of a calculation of the absorption spectrum by CRDS acquired in the case of an absorption-peak measurement for DHO, which is an H₂O isotope, at a gas pressure of 1013.25 Pa (=0.01 atm). FIG. 6B is a pie chart showing a breakdown of the cause of the absorption at a wavenumber indicated by the downward arrow in FIG. 6A (which is the position of the absorption peak of DHO). On the other hand, FIG. 7A is a graph showing the result of a calculation of the absorption spectrum by CRDS acquired in the case of an absorption-peak measurement for DHO, which is an H₂O isotope, at a gas pressure of 101325 Pa (=1 atm), which is 100 times the value used in the case of FIGS. 6A and 6B. FIG. 7B is a pie chart showing a breakdown of the cause of the absorption at a wavenumber indicated by the downward arrow in FIG. 7A (which is the same wavenumber as indicated by the downward arrow in FIG. 6A).

For the calculation, it was assumed that the temperature was 353 K, and the H₂O concentration was 0.03. It was also assumed that the H₂O isotopes contained in the measurement-target gas were introduced into the measurement cell 40 with their natural isotope abundance ratio.

As shown in FIG. 6B, when the gas pressure is 1013.25 Pa, the absorption by DHO, which is one of the H₂O isotopes, forms 93% of the entire absorption at the wavenumber of the absorption peak of DHO. On the other hand, as shown in FIG. 7B, increasing the gas pressure to a higher level which equals 100 times the aforementioned pressure considerably increases the degree of overall absorption, while dramatically decreasing the percentage of the absorption by DHO to 2%, with the absorption almost entirely caused by the H₂O isotopes other than DHO. In this case, since the absorption by DHO cannot be entirely ignored, the background should be removed as in Condition 2 in the previously described example to determine an accurate absolute concentration of the target component, DHO.

Evidently, the device and method according to the present invention are effective for other various kinds of isotope gases in determining the concentration of an isotope having a particularly low concentration.

It should be noted that any of the previous embodiments is a mere example of the present invention, and any change, modification, addition or the like appropriately made within the spirit of the present invention will evidently fall within the scope of claims of the present application.

### REFERENCE SIGNS LIST

1 . . . Laser Source Unit
2 . . . Laser Driver
3 . . . Optical Switch
4 . . . Optical Resonator
40 . . . Measurement Cell
41 . . . Gas Introduction Tube
42 . . . Introduction Valve
43 . . . Gas Discharge Tube
44 . . . Discharge Valve
45 . . . Vacuum Pump
46 . . . Pressure Sensor
47, 48 . . . Mirror
5 . . . Photodetector
6 . . . Control Unit
61 . . . Measurement Controller
62 . . . Laser Controller
63 . . . Pressure Controller
64 . . . Measurement Parameter Storage Section
7 . . . Data Processing Unit
71 . . . Measured Data Storage Section
72 . . . Ring-Down Time Calculator
73 . . . Concentration-Computing Operator
74 . . . Known Information Storage Section for Calculation
8 . . . Output Unit

1. A gas measurement method for determining a concentration of a target component contained in a measurement-target gas by cavity ring-down absorption spectroscopy, comprising:
a first measurement step for performing a measurement by cavity ring-down absorption spectroscopy for a wavelength of an absorption peak of the target component under a first pressure by irradiating the measurement-target gas with laser light;
a second measurement step for performing a measurement by cavity ring-down absorption spectroscopy by irradiating the measurement-target gas under a second pressure different from the first pressure with laser light; and
a calculation step for calculating the concentration of the target component by performing a calculation on a measurement result of the first measurement step and a measurement result of the second measurement step.

2. The gas measurement method according to claim 1, wherein:
the second measurement step is for performing the measurement by cavity ring-down absorption spectroscopy for a wavelength at which an influence of an absorption by the target component is negligible, the wavelength being different from the wavelength of the absorption peak of the target component; and
the calculation step is for estimating a concentration of non-targeted components in the measurement-target gas under the second pressure based on the measurement result of the second measurement step, then estimate, from that concentration, a contribution of an absorption by the non-targeted components to an absorption coefficient determined from the measurement result of the first measurement step, and perform a calculation which removes an influence of the absorption by the non-targeted components.

3. The gas measurement method according to claim 1, wherein:
the second measurement step is for performing the measurement by cavity ring-down absorption spectroscopy for the wavelength of the absorption peak of the target component; and
the calculation step is for preparing a system of equations based on the measurement result of the first measurement step and the measurement result of the second measurement step, and to solve the system of equations to calculate the concentration of the target component from or in which an influence of the non-targeted components in the measurement-target gas is removed or reduced.

4. The gas measurement method according to claim 1, wherein:
the measurement-target gas contains CO₂ gas; and
the target component is ¹⁴CO₂ which is one of isotopes in the CO₂.

5. A gas measurement device configured to determine a concentration of a target component contained in a measurement-target gas by cavity ring-down absorption spectroscopy, comprising:
a laser light emitter;
an optical resonator including a measurement cell configured to contain a measurement-target gas, the optical resonator configured to produce oscillations of laser light emitted from the laser light emitter and introduced into the measurement cell;
a photodetector configured to detect laser light extracted from the optical resonator;
a pressure regulator configured to regulate a pressure of the measurement-target gas in the measurement cell;
a controller configured to control the pressure regulator when performing a measurement for the measurement-target gas in the measurement cell by cavity ring-down absorption spectroscopy; and
a calculation processor configured to calculate the concentration of the target component by performing a calculation on a plurality of measurement results respectively obtained at different pressures under a control of the controller.

6. The gas measurement device according to claim 5, wherein the controller is configured to control the laser emitter and the photodetector in addition to the pressure regulator so as to perform:
a first measurement step for performing a measurement by cavity ring-down absorption spectroscopy for a wavelength of an absorption peak of the target component under a first pressure by irradiating the measurement-target gas with laser light; and
a second measurement step for performing a measurement by cavity ring-down absorption spectroscopy by irradiating the measurement-target gas under a second pressure different from the first pressure with laser light.

7. The gas measurement device according to claim 6, wherein:
the controller is configured to perform, in the measurement under the second pressure, the measurement by cavity ring-down absorption spectroscopy for a wavelength at which an influence of an absorption by the target component is negligible, the wavelength being different from the wavelength of the absorption peak of the target component; and
the calculation processor is configured to estimate, based on a measurement result obtained under the second pressure, a concentration of non-targeted components in the measurement-target gas under the second pressure, then estimate, from that concentration, a contribution of an absorption by the non-targeted components to an absorption coefficient determined from a measurement result obtained under the first pressure, and perform a calculation which removes an influence of the absorption by the non-targeted components.

8. The gas measurement device according to claim 6, wherein:
the controller is configured to perform the measurement by cavity ring-down absorption spectroscopy for the wavelength of the absorption peak of the target component in the measurement under the second pressure; and
the calculation processor is configured to prepare a system of equations based on a measurement result obtained under the first pressure and a measurement result obtained under the second pressure, and to solve the system of equations to calculate the concentration of the target component from or in which an influence of the non-targeted components in the measurement-target gas is removed or reduced.

9. The gas measurement device according to claim 6, wherein the pressure regulator is configured to regulate the pressure of the measurement-target gas in the measurement cell to the first pressure by compulsorily discharging a portion of the measurement-target gas from the measurement cell to an outside, starting from a state in which the measurement cell contains the measurement-target gas under the second pressure.

10. The gas measurement device according to claim 6, wherein the pressure regulator is configured to regulate the pressure of the measurement-target gas in the measurement cell to the second pressure by additionally supplying the measurement cell with the measurement-target gas which remains unsupplied, starting from a state in which the measurement cell is filled with the measurement-target gas supplied beforehand and contains the measurement-target gas under the first pressure.
